(19) United States
(12) Patent Application Publication
(10) Pub. No.: US 2024/0238186 A1
(43) Pub. Date: Jul. 18, 2024

(54) Topical Compositions Comprising (Glyco)Sphingolipids and/or (Glyco)Ceramides

(71) Applicant: CarboCode S.A., Cantanhede (PT)

(72) Inventors: Bruce McConnell, Cantanhede (PT); Cornelia Pyko, Cantanhede (PT); Feryel Adam, Cantanhede (PT)

(73) Assignee: CarboCode S.A., Cantanhede (PT)

(21) Appl. No.: 18/557,661

(22) PCT Filed: Apr. 29, 2022
(86) PCT No.: PCT/PT2022/050018

§ 371 Date: Oct. 27, 2023

(30) Foreign Application Priority Data
Apr. 30, 2021 (CH) 00470/21

(51) Int. Cl. A61K 8/68; A61K 31/164; A61K 31/7028; A61Q 17/00; A61Q 19/00; A61Q 19/08

(52) U.S. Cl. CPC A61K 8/68; A61K 31/164; A61K 31/7028; A61Q 17/00; A61Q 19/007; A61Q 19/08

(57) ABSTRACT

The present invention relates to a topical composition comprising one or more compounds of formula (I), wherein X is hydrogen or a glycosyl moiety, the carbon-carbon bond noted [symbol] is a double or a single bond, R¹ is an alkyl chain having 10-20 carbon atoms, R² is hydrogen or a substituted or unsubstituted acyl having 16-34 carbon atoms, R³ is hydrogen when the carbon-carbon bond noted [symbol] is a double or a single bond, and R³ is —OH when the carbon-carbon bond noted [symbol] is a single bond, R⁴ is hydrogen when the carbon-carbon bond noted [symbol] is a double or a single bond, and R⁴ is hydrogen or —OH when the carbon-carbon bond noted [symbol] is a double bond, said composition is characterized in that at least one of the one or more compounds of formula (I) has a lactosyl moiety or a glucosyl moiety in the position X.

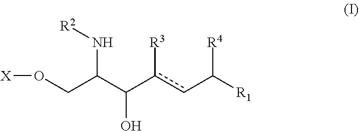

### FIELD OF THE INVENTION

The present invention relates to a topical composition and to a cosmetic or medical use of a topical composition.

### BACKGROUND OF THE INVENTION

The skin is a critical barrier for the body. It prevents the loss of water and electrolytes and acts as a barrier to external assaults on the body, such as from ultra-violet light, pollution, infectious agents, etc. The epidermis, or outer portion of the skin, provides the primary barrier function of the skin. The epidermis is made up of layers of which the stratum corneum is the outermost layer and the stratum basale is the innermost layer. The epidermis contains no blood vessels, and cells in the deepest layers are nourished by diffusion from blood capillaries extending to the upper layers of the dermis.

The stratum basale of the epidermis contains keratinocytes which proliferate through mitosis. The daughter cells move up through the layers of the epidermis, changing shape and composition as they undergo multiple stages of cell differentiation. The daughter cells eventually become terminally differentiated when they reach the stratum corneum. Within the stratum corneum, the terminally differentiated keratinocytes are referred to as corneocytes. The corneocytes are filled with water-retaining keratin proteins and are attached together through corneodesmosomes. Corneocytes do not contain a nucleus. The corneocytes are eventually shed from the surface of the skin, a process called desquamation.

The stratum corneum acts as the main barrier in the skin. It is made up of about 10 to 30 layers of corneocytes surrounded by lipid layers (lipid lamellae). The stratum corneum is thus described as a wall composed of bricks (corneocytes) and mortar (lipids). The main constituents of the lipid layers are ceramides, cholesterol, and free fatty acids in a 1:1:1 molar ratio. The ceramides play the most important role in the barrier function of the stratum corneum. Disruption or decrease of ceramides in the stratum corneum weakens the skin barrier function, thereby e.g. increasing transdermal water loss. Many conditions of the skin such as atopic dermatitis, psoriasis, contact dermatitis, etc are associated with alterations in the ceramide composition of the stratum corneum. Also, alterations in the ceramide composition of the stratum corneum can lead to skin conditions, such as dehydration of the skin. This manifests in skin which appears wrinkled, harsh, and rough, which has impaired desquamation and which has lost its elasticity. Age and extrinsic assaults appear to be key factors in alteration or decline of ceramide composition. Consequently, improving the ceramide composition of the stratum corneum has been a key objective of the cosmetic and skin care industry.

Improvement of the ceramide composition of the skin has generally involved incorporation of ceramides or pseudo-ceramides into topical skincare formulations. Unfortunately, ceramides are highly insoluble and have high melting points. This makes formulation difficult. Also, their tendency to recrystallise complicates their stable incorporation into topical formulations.

Efforts to overcome formulation difficulties have generally focused on combining ceramides with other lipids. For example, WO1999029293 describes a combination of a free sphingoid base and a ceramide. Alternatively, WO2018177730 describes melting a mixture of, or a wax and a ceramide, and then incorporating the melt into an oil-containing formulation.

Another approach has been topical application of a fraction of bovine milk fat globule membrane on the skin. In particular, application of a milk sphingolipid-enriched fraction (MSEF) extracted from the milk fat globule membrane found in sweet buttermilk powder improved the water holding capacity and skin barrier recovery of damaged skin (Lee et al, Int J. Cos. Sci., 2020). The main sphingolipids found in the MSEF fraction were bovine glucosylceramide, lactosylceramide and sphingomyelin.

An alternative approach to modulate the ceramide composition of the stratum corneum is to apply a composition which induces epidermal lipid production and ceramides in particular. For example, application of an oat oil extract on keratinocytes is reported to increase lipid synthesis including ceramides; potentially through activation of peroxisome proliferator-activated receptors (Chon et al, Exp. Dermatol, 2015).

However, there remains a need for topical compositions that comprise ceramide species that are stereochemically and structurally identical to those found in the human stratum corneum.

### SUMMARY OF THE INVENTION

The present invention relates to topical compositions comprising (glyco)sphingolipids and/or (glyco)ceramides that can be used in cosmetic and medical applications.

Specifically, the present invention comprises the following aspects:
(1) A topical composition comprising one or more compounds of formula I

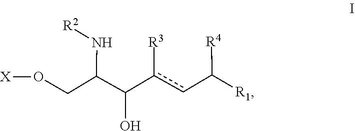

wherein
X is hydrogen or a glycosyl moiety,
the carbon-carbon bond noted [symbol] is a double or a single bond,
R¹ is an alkyl chain having 10-20 carbon atoms,
R² is hydrogen or a substituted or unsubstituted acyl having 16-34 carbon atoms,
R³ is hydrogen when the carbon-carbon bond noted [symbol] is a double or a single bond, and R³ is —OH when the carbon-carbon bond noted [symbol] is a single bond,
R⁴ is hydrogen when the carbon-carbon bond noted [symbol] is a double or a single bond, and R⁴ is hydrogen or —OH when the carbon-carbon bond noted [symbol] is a double bond, said composition is characterized in that at least one of the one or more compounds of formula I has a lactosyl moiety or a glucosyl moiety in the position X.
Topical compositions comprising one or more compounds of formula I administered onto skin of an individual for a period of time
   increase the ceramide content of the stratum corneum,
   increase keratinocyte terminal differentiation in the epidermis,
   upregulate enzymes in the epidermis associated with epidermal ceramide synthesis, and/or
   improve skin barrier function in said individual.
(2) Cosmetic use of one or more compounds of formula I or topical compositions comprising thereof. Beneficial effects cosmetic treatment with topical compositions comprising one or more compounds of formula I are one or more of following
   Improving skin complexion;
   Improving skin radiance;
   Improving skin tone;
   Skin soothing;
   Reducing premature skin ageing;
   Wrinkle smoothing and/or
   Supporting healthy look of skin.
(3) Pharmaceutical topical compositions comprising one or more compounds of formula I for use in treating a skin pathological condition or a skin disease in an individual, such as acne, ichthyosis, xeroderma, eczema, atopic dermatitis and/or psoriasis.
(4) Method for non-pharmacological management of a skin condition associated with an impaired skin barrier function, comprising topically administering a composition comprising one or more compounds of formula I onto skin of an individual.
(5) Method for non-pharmacological intervention for mitigation of a skin condition or a skin disease in an individual who has, or who is susceptible to developing an impaired skin barrier function, the method comprising topically administering a composition comprising one or more compounds of formula I onto skin of an individual.

Various embodiments relating to the above aspects of the invention are described throughout specification of the invention and illustrated by non-limiting examples.

### FIGURES

FIGS. 1 to 5 show heatmaps using a log 2 scale with up- or downregulation of respective proteins upon treatment with 0,01% (01), 0,03% (03) and 0,15% (15) of respective ceramide in vehicle as compared to vehicle without ceramide (VEH) (FIG. 1: LacCerNS, FIG. 2: GlcCerNS, FIG. 3: CerNP. FIG. 4: CerNG, FIG. 5: CerNS); detailed experimental setup explained in Example 4.

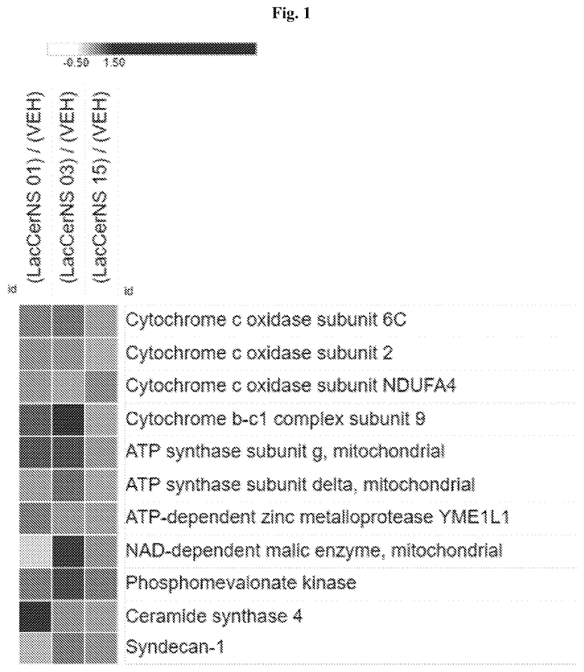

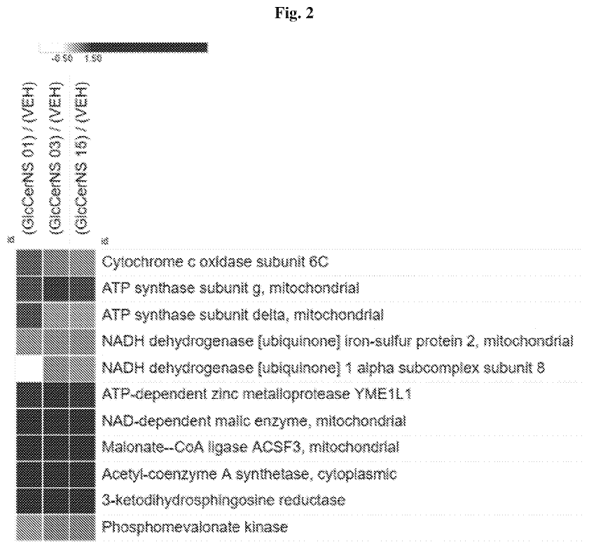

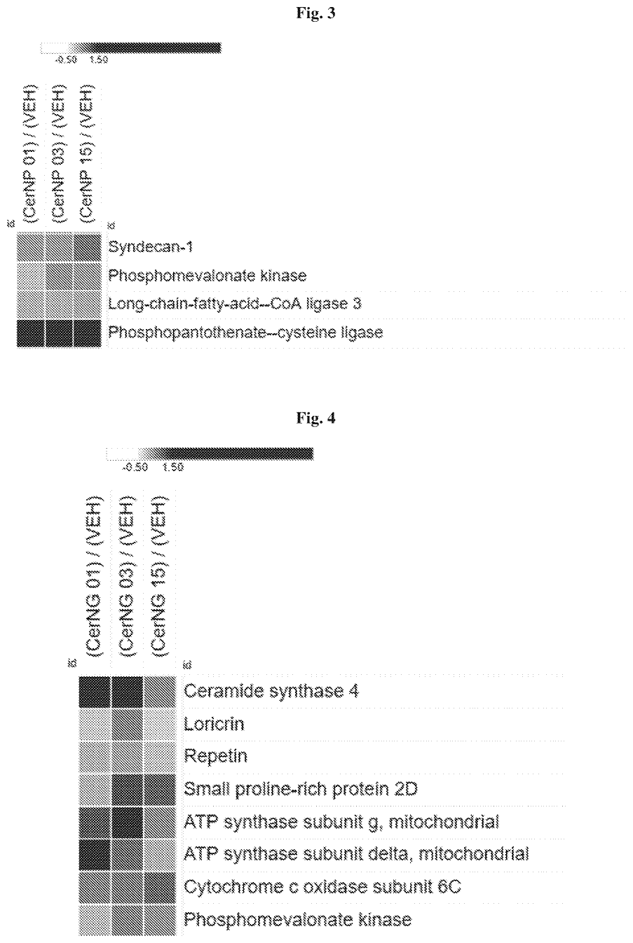

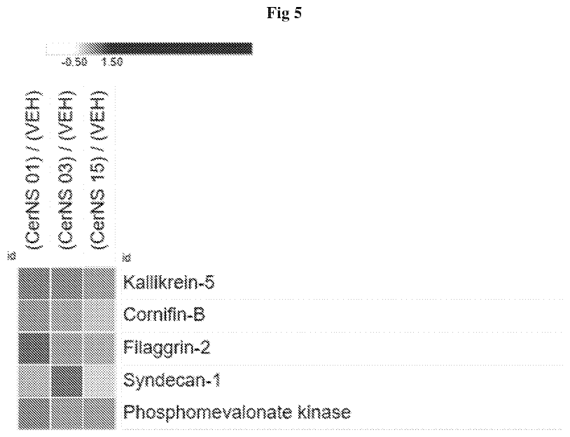

### DETAILED DESCRIPTION OF THE INVENTION

It has now been surprisingly found that topical administration of one or more compounds of formula I onto the skin, increases the concentration of ceramide in the stratum corneum. Without to be bound to a theory, it is believed that the one or more compounds of formula I may achieve this by upregulating enzymes in the epidermis associated with epidermal ceramide synthesis. Further, one or more compounds of formula I advantageously increase keratinocyte terminal differentiation in the epidermis and improve skin barrier function in skin of an individual. In this specification, the following terms have the following meanings:

“Topical composition” includes compositions suitable for topical application on keratinous tissue, especially on skin. Such compositions are typically dermatologically acceptable in that they do not have undue toxicity, incompatibility, instability, allergic response, and the like, when applied to skin. Topical compositions typically comprise a topically acceptable carrier.

Topical skin care compositions of the present invention can have a selected viscosity to avoid significant dripping or pooling after application to skin. Accordingly, a topical cosmetic or medical composition of the invention comprising one or more compounds described herein, would typically comprise one or more other compounds that are useful for formulating these compositions. Typically, such “formulating” compounds do not possess the biological activity of the compounds of the invention.

“Keratinous tissue” includes keratin-containing layers disposed as the outermost protective covering of mammals and includes, but is not limited to, lips, skin, hair and nails.

The terms “about” or “approximately” are defined as being close to the value or range following said terms, as understood by one of ordinary skill in the art, and include a deviation of up to 10% of the values or ranges that follow.

The term “reducing” or any variation of the term such as “reduction” includes any measurable decrease to achieve a desired effect.

The terms “promoting”, “enhancing”, “improving” or “increasing” or any variation of these terms include any measurable increase to achieve a desired effect.

“Effective amount” means an amount sufficient to render a desired treatment or management outcome in a human. An effective amount can be administered in one or more doses to achieve the desired treatment or management outcome.

“Preventive treatment”, “preventing” or “prevention” means treatment given or action taken to diminish the risk of onset or recurrence of a cosmetic or medical condition, or of an effect related to an impairment in the visual appearance of a skin or of a skin disease.

“Primary prevention” means prevention of the initial onset of a cosmetic or a medical condition in an individual.

“Secondary prevention” means, in an individual who has a cosmetic or a medical condition or who has had a condition, (i) prevention of reoccurrence of the condition, (ii) increase in the duration of remission of the condition, and/or (iii) reduction in severity of symptoms of the condition.

“Treat” means to address a cosmetic or a medical condition or disease with the objective of improving or stabilising an outcome in the person being treated or addressing an underlying need. Treat therefore includes the topical management of the cosmetic or medical condition or disease by addressing dermatological needs of the person being treated. “Treating” and “treatment” have grammatically corresponding meanings.

“Therapy” means treatment given or action taken to reduce or eliminate symptoms of a disease or pathological condition.

A child as referred to herein denotes a human individual until the age of 12.

A teenager as referred to herein denotes a human individual having an age of between 13 and 19.

The above term definitions and embodiments described throughout of the specification are applicable to all aspects of the invention, unless specified otherwise.

In one aspect, the present invention relates to a topical composition comprising one or more compounds of formula I

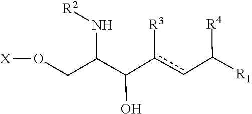

wherein
X is hydrogen or a glycosyl moiety, the carbon-carbon bond noted [symbol] is a double or a single bond,
R¹ is an alkyl chain having 10-20 carbon atoms,
R² is hydrogen or a substituted or unsubstituted acyl having 16-34 carbon atoms,
R³ is hydrogen when the carbon-carbon bond noted [symbol] is a double or a single bond, and R³ is —OH when the carbon-carbon bond noted [symbol] is a single bond,
R⁴ is hydrogen when the carbon-carbon bond noted [symbol] is a double or a single bond, and R⁴ is hydrogen or —OH when the carbon-carbon bond noted [symbol] is a double bond,
said composition is characterized in that at least one of the one or more compounds of formula I has a lactosyl moiety or a glucosyl moiety in the position X.

As used herein, the term “alkyl” refers to an acyclic straight hydrocarbyl group in which the carbon atoms may be saturated or contain one or more double and/or triple bonds (so, forming for example an alkenyl or an alkynyl). Examples of “alkyl” include, but are not limited to, methyl, ethyl, n-propyl, isopropyl, isobutyl, n-butyl, see-butyl, tert-butyl, isopentyl, n-pentyl, neo-pentyl, n-hexyl, ethenyl, propenyl, 1-butenyl, 2-butenyl, isobutenyl, 1-pentenyl, 2-pentenyl, 2-methyl-1-butenyl, 3-methyl-1-butenyl, 2-methyl-2-butenyl, 1-hexenyl, 2-hexenyl, 3-hexenyl, methylpentenyl, dimethylbutenyl, ethynyl, propynyl, 1-butynyl, 2-butynyl, pentynyl, and hexynyl. Typically, the term alkyl refers to a saturated hydrocarbyl group.

As used herein, the term “acyl” includes both saturated groups and groups having one or more double and/or triple bonds. In the context of acyls, the term “substituted” means that the carbon atoms may be substituted one or several times, preferably 1 to 3 times, with functional group(s).

In a preferred embodiment, R² of compound I is an unsubstituted acyl group. In a more preferred embodiment, R² of compound I or II is a saturated fatty acyl group or a fatty acyl group having one or more double bonds.

The compounds of formula I may be obtained by any technique known to the skilled person. The compounds of formula I may be obtained by a fermentation technique or by a biocatalytic process. For example, glycosphingoplipids such as lactosylceramide may be produced starting from the respective glycosyl fluoride, such as lactosyl fluoride, followed by coupling to a (acylated) sphingoid base by the use of an endoglycoceramide synthase, such as described in Rich et al., ChemComm (2011) 47:10806-10808. Sphingoid bases may be produced by a fermentation process, such as e.g. described in WO 95/12683 A1, especially phytosphingosine or a precursor thereof (claim 1 of EP 0 726 960 B1).

In some embodiments, the topical composition comprises one or more compounds of formula I, wherein the residue X is lactosyl.

In some embodiments, the topical composition comprises at least two compounds of formula I, wherein for one compound of formula I the residue X is lactosyl and for the other compound of formula I the residue X is galactosyl.

In some embodiments, the topical composition comprises at least two compounds of formula I, wherein for one compound of formula I the residue X is lactosyl and for the other compound of formula I the residue X is hydrogen.

In some embodiments, the topical composition comprises at least two compounds of formula I, wherein for one compound of formula I the residue X is lactosyl and for the other compound of formula I the residue X is glucosyl.

In some embodiments, the topical composition comprises at least three compounds of formula I, wherein for one compound of formula I the residue X is lactosyl for the second compound of formula I the residue X is galactosyl and for the third compound of formula I the residue X is glucosyl.

In some embodiments, the topical composition comprises at least three compounds of formula I, wherein for one compound of formula I the residue X is lactosyl, for the second compound of formula I the residue X is galactosyl, and for the third compound of formula I the residue X is hydrogen.

In some embodiments, the topical composition comprises at least three compounds of formula I, wherein for one compound of formula I the residue X is lactosyl, for the second compound of formula I the residue X is hydrogen, and for the third compound of formula I the residue X is glucosyl.

In some embodiments, the topical composition comprises at least four compounds of formula I, wherein for one compound of formula I the residue X is lactosyl for the second compound of formula I the residue X is hydrogen, for the third compound of formula I the residue X is glucosyl, and for the fourth compound of formula I the residue X is galactosyl.

In some embodiments, for one or more compounds of formula I of the topical composition, R¹ is —C₁₂H₂₅ and R² is hexadecanoyl, 2-hydroxyhexadecanoyl, octadecanoyl, 2-hydroxyoctadecanoyl, eicosanoyl, 2-hydroxyeicosanoyl, lignoceroyl, 2-hydroxytetracosanoyl or 30-(linoleyloxy)-triacontanoyl, preferably octadecanoyl or lignoceroyl.

The one or more compounds of formula I may be formulated into topical compositions suitable for application to the skin. Generally, the topical compositions of the present invention will contain from about 0,0001% to about 10% wt/wt of a compound of formula I, such as from about 0,005% to about 5% wt/wt of a compound of formula I, or from 0,01% to about 1% wt/wt of a compound of formula I. For example, the topical compositions of the present invention may contain from about 0,0001 to about 0,00025% wt/wt of a compound of formula I, from about 0,0002 to about 0,0004% wt/wt of a compound of formula I, from about 0,0003 to about 0,0005% wt/wt of a compound of formula I, from about 0,0004 to about 0,0006% wt/wt of a compound of formula I, from about 0,0005 to about 0,0008% wt/wt of a compound of formula I, from about 0,0007 to about 0,001% wt/wt of a compound of formula I, from about 0,001 to about 0,005% wt/wt of a compound of formula I, from about 0,003 to about 0,008% wt/wt of a compound of formula I, from about 0,005 to about 0,01% wt/wt of a compound of formula I, from about 0,008 to about 0,05% wt/wt of a compound of formula I, from about 0,05 to about 0,08% wt/wt of a compound of formula I, from about 0,06 to about 0,1% wt/wt of a compound of formula I, from about 0,08 to about 0,1% wt/wt of a compound of formula I, etc.

In some embodiments, the topical compositions of the present invention contain an amount of about 0,01% to 5% wt/wt of a compound of formula I. or an amount of about 0,02% wt/wt to 1% wt/wt of a compound of formula I. Examples are 0,05% wt/wt, 0,1% wt/wt or 0,2% wt/wt of a compound of formula I. Compounds of formula I in which the residue X is a glycosyl moiety selected from lactosyl, glucosyl and galactosyl may be present in an amount of 0,1% wt/wt. Compounds of formula I in which the residue X is hydrogen may be present in an amount of 0,2% wt/wt.

In other embodiments, the topical compositions can contain an amount of about 5% to 10% wt/wt of a compound of formula I.

The topical compositions according to the present invention may comprise a single compound of formula I, e.g. a compound comprising the lactosyl moiety in position X of formula I, in the ranges or amounts as listed above.

When the topical composition according to the present invention comprises more than one compound of formula I, also termed herein as blend of compounds of formula I, each compound of formula I may be present in the same amount or in different amounts. The ranges or amounts as listed above may relate to the total content of the blend in the topical composition, or to the amount of one compound in the blend in the topical composition. For example, in some embodiments, the topical composition may comprise a blend of compounds of formula I with a total content of the blend of 0,1% wt/wt, wherein the blend has glucosylceramide in an amount of 0,02% wt/wt and lactosylceramide in an amount of 0,08% wt/wt. In some embodiments, the topical composition may comprise a blend of compounds of formula I with a total content of the blend of 0,2% wt/wt, wherein the blend has lactosylceramide in an amount of 0,04% wt/wt and ceramide NP in an amount of 0,16% wt/wt. In some embodiments, the topical composition may comprise a blend of compounds of formula I with a total content of the blend of 0,2% wt/wt, wherein the blend has lactosylceramide in an amount of 0,04% wt/wt and ceramide NS in an amount of 0,16% wt/wt. In some embodiments, the topical composition may comprise a blend of compounds of formula I with a total content of the blend of 0,2% wt/wt, wherein the blend has lactosylceramide in an amount of 0,04% wt/wt and ceramide NG in an amount of 0,16% wt/wt. Non-limiting exemplary embodiments are also depicted in Example 1.

In other embodiments, the topical composition may comprise a blend of compounds of formula I, wherein X is hydrogen. For example, in some embodiments, the topical composition may comprise a blend of compounds of formula I with a total content of the blend of 0,1% wt/wt, wherein the blend has ceramide NP in an amount of 0,06% wt/wt, ceramide NS in an amount of 0,02% wt/wt and ceramide NG in an amount of 0,02% wt/wt. In some embodiments, the topical composition may comprise a blend of compounds of formula I with a total content of the blend of 0,2% wt/wt, wherein the blend has ceramide NP in an amount of 0,11% wt/wt, ceramide NS in an amount of 0,04% wt/wt and ceramide NG in an amount of 0,05% wt/wt. In some embodiments, the topical composition may comprise a blend of compounds of formula I with a total content of the blend of 0,5% wt/wt, wherein the blend has ceramide NP in an amount of 0,28% wt/wt, ceramide NS in an amount of 0,09% wt/wt and ceramide NG in an amount of 0,13% wt/wt. In some embodiments, the topical composition may comprise a blend of compounds of formula I with a total content of the blend of 0,1% wt/wt, wherein the blend has ceramide NP in an amount of 0,075% wt/wt, ceramide NS in an amount of 0,02% wt/wt and ceramide NG in an amount of 0,005% wt/wt. In some embodiments, the topical composition may comprise a blend of compounds of formula I with a total content of the blend of 0,2% wt/wt, wherein the blend has ceramide NP in an amount of 0,15% wt/wt, ceramide NS in an amount of 0,04% wt/wt and ceramide NG in an amount of 0,01% wt/wt. In some embodiments, the topical composition may comprise a blend of compounds of formula I with a total content of the blend of 0,2% wt/wt, wherein the blend has ceramide NP in an amount of 0,1% wt/wt, ceramide NS in an amount of 0,05% wt/wt and glucosylceramide in an amount of 0,05% wt/wt. Non-limiting exemplary embodiments are also depicted in Example 2.

The amount of the one or more compounds of formula I in a composition of the invention may vary depending upon factors such as the age of the person, the risk and severity of any underlying condition, the environment the person is exposed to, the form of the topical composition, its use for cosmetic or medical purposes, and any other skin care compositions being administered. Appropriate doses for any particular person or indication may be determined by methods known to skin care practitioners. The dose may also vary depending upon whether the topical composition is a “leave on” or “rinse off” formulation with “rinse off” formulations generally containing lower doses. Further, the dose may vary depending on whether the topical composition is applied in an intervention treatment phase or in a maintenance phase. Generally higher doses can be applied in an initial intervention phase than in a maintenance phase.

Topical composition according to the present invention may have a pH in the range of about 4.5 to about 8, e.g. from about 4.5 to about 6.

Topical compositions of the present invention can be structured or formulated into a variety of different forms. Non-limiting examples include emulsions (e.g., water-in-oil, water-in-oil-in-water, oil-in-water, silicone-in-water, water-in-silicone, oil-in-water-in-oil, oil-in-water-in-silicone emulsions), creams, lotions, solutions (both aqueous and hydroalcoholic), anhydrous bases (such as lipsticks and powders), gels, masks, peelings, and ointments. Variations and other structures will be apparent to the skilled artisan and are appropriate for use in the present invention.

In some embodiments, a topical composition may be formulated as an emulsion.

A topical composition of the present invention is typically intended for application on the face, on the body and/or on the hair. The topical composition may be for application on the face or on the body.

A topical composition of the present invention may comprise a compound of formula I, which is selected from the group consisting of ceramide NS (CerNS or CNS), ceramide NG (CerNG or CNG), ceramide NP (CerNP or CNP), glucosyl ceramide or lactosyl ceramide, or a combination thereof.

Ceramide NS, ceramide NdS and ceramide NP are named according to the shorthand nomenclature developed by Motta et al., Biochim Biophys Acta., 1993, 1182:147-151 and expanded by Rabionet et al., Biochim Biophys Acta, 2014, 1841:422-434, and by Masukawa et al., Journal of Lipid Research, 2008, 49, 1466-1476. Ceramides NdS may also be referred to as ceramide NG. For each species, the number of carbons and unsaturations (if present) may be expressed in parentheses following the letters of N, A, E, and O. When not specified, the carbon chain length of the sphingoid base moiety of the ceramides mentioned herein is typically C₁₈. When not specified, the carbon chain length of the acyl moiety of the ceramides mentioned herein may be e.g. C₁₆, C₁₈, C₂₀, C₂₂ or C₂₄.

The glucosyl ceramide and the lactosyl ceramide as mentioned herein are preferably ceramide NS, typically with an acyl chain length of C₁₈. Glucosyl ceramide which is ceramide NS and has an acyl chain length of Cis may be referred to herein also as GlcCNS. Lactosyl ceramide which is ceramide NS and has an acyl chain length of C₁₈ may be referred to herein also as LacCNS.

According to the invention, application of a topical composition described herein on skin of an individual increases the ceramide content of the stratum corneum, increases keratinocyte terminal differentiation in the epidermis, upregulates enzymes in the epidermis associated with epidermal ceramide synthesis and/or improves skin barrier function in said individual. These effects may be determined by an upregulation of respective differentiation markers in vitro. For example, an increase of keratinocyte terminal differentiation in the epidermis may be determined by an upregulation of differentiation markers such as loricrin, filaggrin or involucrin. Upregulation of enzymes in the epidermis associated with epidermal ceramide synthesis may be e.g. measured in vitro in keratinocytes as explained in Example 4.

The effects may also be measured with further other suitable standard technique known to the skilled person. An increase of the ceramide content of the stratum corneum can be e.g. determined with the tape stripping procedure as e.g. described in Lademann et al., European Journal of Pharmaceutics and Biopharmaceutics (2009) 72:317-323. An improvement of skin barrier function may be e.g. determined by measuring TEWL and skin hydration with a corneometer or tewameter.

Accordingly, a topical composition of the present invention may advantageously be used for cosmetic treatment or medical treatment of keratinous tissue, especially skin, of an individual. In the context of the use of the inventive topical composition for cosmetic purposes, the topical composition according to the invention may be referred to as topical cosmetic composition or cosmetic composition. Cosmetic treatment is herein defined as to improve the visual appearance of skin of the treated individual. By the wording “improve visual appearance of skin” is meant that cosmetic treatment with a topical composition of the invention comprising one or more compounds of formular I, in particular comprising one or more of the following beneficial effects on the treated skin of an individual:
reducing skin roughness.
reducing skin flakiness.
reducing the depth of wrinkles in the skin.
strengthening the skin elasticity.
increasing the tautness of the skin.
increasing the thickness of the skin.
increasing skin moisture.
calming irritated skin
normalizing and improving the skin tone.

All the above effects have a visual impression of skin as radiant. i.e. the glowy, vibrant look associated with healthy, well-rested skin. Skin tone, luminosity, firmness, and discolouration (think dark circles and sun damage) can all have an effect on the natural appearance of radiant skin.

In particular, the cosmetic compositions of the invention preferably
improve skin complexion, i.e, color and texture of the skin, especially on the face;
improve skin radiance, i.e. skin tone, luminosity, firmness, and discolouration (think dark circles and sun damage);
improve skin tone, i.e. normalizing natural hue of the skin, reducing redness and reducing pigmentation;
soothe the skin, i.e, calming irritated skin, e.g. mitigating, assuaging, or allaying pain from sunburning or dehydrating of the skin
reduce the signs of skin ageing, both chronological and premature, e.g the signs of ageing due to exposure of the skin to intrinsic and extrinsic factors, like decrease in production of essential molecules supporting the skin natural structure, loss of water, UV light damage, or exposure to chemical internal radicals generated in the body (due to unhealthy eating or life style, a disease, etc) or external pollutants hazardous for the skin; and/or
smooth skin wrinkles, in particular, the face skin wrinkles, both fine surface lines and deeper furrows, i.e. lessen the appearance of wrinkled skin, smoothing the skin surface;

In general, cosmetic compositions of the invention support and enhance healthy look of the skin, especially of the face and neck skin.

In some preferred embodiments, the skin may be an aged and/or a dry skin. The cause of dryness of the skin may be of constitutional or acquired type.

In the case of acquired dry skin, the involvement of outside parameters such as exposure to chemical agents, to difficult climatic conditions or to sunlight, alternatively certain therapeutic treatments (retinoids, for example) is determinant. Under these outside influences, the skin may then become momentarily and locally dry. Dry skin is a common condition that is attributed to a lack of water in the stratum corneum and to an impaired synthesis of natural moisturizing factor (NMF).

Non-pathological acquired constitutional dry skin can be characterized by a deficiency in lipids constituting the barrier and/or the aqueous-lipid film, and/or an endogenous insufficiency of sebum production by the sebaceous glands.

Skin aging is a complex phenomenon responsible for progressive changes of the skin. Many factors contribute to skin aging and associated alterations in appearance and function of the skin, such as the actual age of a person, the amount of exposure to environmental factors (e.g., sun light, pollution, chemicals, smoke, etc.), and how well a person has taken care of their skin. In particular, skin aging concerns two processes—intrinsic aging, which is related to the natural aging process and genetic influences, and extrinsic aging, which is accumulated damage due to exposure to environmental factors.

Intrinsic aging process in cells and skin can be related to the loss of proper function of the skin in maintaining biochemical pathways. Such pathways can control the oxidative/reductive environment balance in the skin, the regulation of inflammation, and the maintenance of the moisture balance of the skin. Losses of proper function of the skin can lead to increased oxidative damage, increased inflammation, dry skin, loss of skin firmness, increased skin unevenness, and increased fine lines and wrinkles.

Factors that cause extrinsic aging can include exposure to ultraviolet (UV) rays, irritants, and pollutants, such as Polycyclic aromatic pollutants (PAH), Particulate Matter PM2.5 (micrometers), blue light, Ozone, or cigarette smoke. UV rays, through sun exposure or the use of ultraviolet lamps (for example, tanning beds), can induce oxidative stress, inflammation, production of melanin, and even genetic mutations that leads to skin damage. The accumulation of oxidative stress through free radical formation can damage skin proteins leading to skin aging, which includes loss of elasticity, loss of dermal proteins, lines and wrinkles, and abnormal pigmentation. Similarly, accumulation of fine particles such as PM 2.5 (fine particles that have an aerodynamic diameter under 2.5 pm) on the skin can also induce oxidative stress and inflammation. These fine particles may also cause damage on barrier proteins of the skin, leading to loss of moisture and elasticity of the skin.

The appearance of aged skin targeted by the invention include thinning of the skin, a loss of firmness, a loss of elasticity, a loss of density or a loss of tonicity of the skin, the appearance of a marked microrelief of the skin, the appearance of roughness, the formation and/or presence of fine lines and/or of wrinkles, a modification of the radiance of the skin complexion, a wizened appearance of the skin, sagging of the skin, and/or withering of the skin.

The cosmetic treatment according to the present invention typically has one or more beneficial effects on skin of an individual. These beneficial effects include one or more of improving skin complexion, improving skin radiance, improving skin tone, skin soothing, reducing premature skin aging, wrinkle smoothing and/or supporting healthy look of skin.

Skin soothing or skin calming as used herein denote protection from skin irritation or reduction of skin irritation and typically has a gentle calming effect.

Additionally to the above described biological effects, the present inventors have surprisingly found that application of lactosylceramide on the skin leads to an upregulation of syndecan-1, of ceramide synthase 4 and of phosphomevalonate kinase.

Syndecan-1 is known to play a role in keratinocyte proliferation and differentiation.

Ceramide synthase 4 catalyzes the formation of ceramides.

Phosphomevalonate kinase is involved in cholesterol synthesis.

The upregulation of both ceramide synthase 4 and phosphomevalonate kinase enzymes suggests that topical administration of lactosylceramide helps to provide cholesterol and ceramides to maintain the skin barrier.

Accordingly, the present invention relates to the use of lactosylceramide for increasing the ceramide content of the stratum corneum, increasing keratinocyte terminal differentiation in the epidermis, upregulating enzymes in the epidermis associated with epidermal ceramide synthesis and/or improving skin barrier function in skin of an individual.

The present inventors have surprisingly found that application of ceramide NP on the skin leads to an upregulation of syndecan-1, long-chain-fatty-acid-CoA ligase 3, phosphopantothenate-cysteine ligase and phosphomevalonate kinase.

Long-chain-fatty-acid-CoA ligase 3 activates long-chain fatty acids for synthesis of cellular lipids.

Phosphopantothenate-cysteine ligase catalyzes the second step in the biosynthesis of coenzyme A from vitamin B5.

The supply of essential lipids of the skin's protective barrier is crucial for its function. Coenzyme A is a very important molecule in lipid metabolism, it is converted to acetyl-CoA, which is a precursor in the synthesis of fatty acids, cholesterol and ceramides. Thus, the upregulation of phosphomevalonate kinase, long-chain-fatty-acid-CoA ligase 3 and phosphopantothenate-cysteine ligase involved in these pathways indicates that ceramide NP may help to provide free fatty acids, cholesterol and ceramides to maintain the skin barrier.

Accordingly, the present invention additionally relates to the use of ceramide NP for increasing the ceramide content of the stratum corneum, increasing keratinocyte terminal differentiation in the epidermis, upregulating enzymes in the epidermis associated with epidermal ceramide synthesis and/or improving skin barrier function in skin of an individual.

The present inventors have also surprisingly found that application of ceramide NG on the skin leads to an upregulation of ceramide synthase 4, of loricrin, of repetin, of small proline-rich protein 2D and of phosphomevalonate kinase.

Loricrin is a major keratinocyte cell envelope protein.

Repetin is involved in the cornified cell envelope formation.

Small proline rich protein 2D is a cross-linked envelope protein of keratinocytes.

Cornification is an essential process to build the skin barrier, during which the cell membrane of keratinocytes is replaced by the cornified envelope. The cornified envelope is an agglomeration of several structural proteins that are crosslinked. Loricrin (makes up to 70-80% of protein content of cornified envelope), repetin & small proline-rich protein 2d are part of the cornified envelope and their upregulation indicates that ceramide NG triggers the formation of the cornified envelope and therefore the keratinocyte terminal differentiation and may improve the skin barrier function.

The supply of essential lipids of the skin's protective barrier is crucial for its function. Ceramide Synthase 4 catalyzes the formation of ceramides and phosphomevalonate kinase is involved in cholesterol synthesis. Thus, the upregulation of both enzymes indicates that ceramide NG helps to provide cholesterol and ceramides to maintain the skin barrier.

Accordingly, the present invention in one aspect relates to the use of ceramide NG for increasing the ceramide content of the stratum corneum, increasing keratinocyte terminal differentiation in the epidermis, upregulating enzymes in the epidermis associated with epidermal ceramide synthesis and/or improving skin barrier function in skin of an individual.

Yet, the present inventors have surprisingly found that application of glucosyl ceramide on the skin leads to an upregulation of 3-ketodihydrosphingosine reductase, of acetyl-coenzyme A synthetase (cytoplasmic), of malonate-CoA ligase ACSF3 (mitochondrial) and of phosphomevalonate.

3-ketodihydrosphingosine reductase kinase catalyzes the reduction of 3-ketodihydrosphingosine to dihydrosphingosine.

Acetyl-coenzyme A synthetase catalyzes the synthesis of acetyl-CoA from short-chain fatty acids. Malonate—CoA ligase ACSF3 catalyzes the initial reaction in intramitochondrial fatty acid synthesis, by activating malonate and methylmalonate into their respective CoA thioester.

The supply of essential lipids of the skin's protective barrier is crucial for its function. Acetyl-CoA and Malonyl-CoA are both precursors of the synthesis of fatty acids, cholesterol and ceramides. The Phosphomevalonate kinase is involved in the cholesterol synthesis and 3-ketodihydrosphingosine reductase in the ceramide synthesis. Thus, the upregulation of those enzymes indicates that glucosyl ceramide CNS helps to provide free fatty acids, cholesterol and ceramides to maintain the skin barrier.

Accordingly, in a further aspect the present invention relates to the use of glucosyl ceramide for increasing the ceramide content of the stratum corneum, upregulating enzymes in the epidermis associated with epidermal ceramide synthesis and/or improving skin barrier function in skin of an individual.

Further, the present inventors have surprisingly found that application of ceramide NS on the skin leads to an upregulation of cornifin-B, of filaggrin-2, of syndecan-1, of kallikrein-5 and of phosphomevalonate kinase.

Cornifin-B is a cross-linked envelope protein of keratinocytes.

Filaggrin-2 is important for proper integrity and mechanical strength of the stratum corneum of the epidermis, Essential for normal cell-cell adhesion in the cornified cell layers.

Kallikrein-5 is a protease that cleaves corneodesmosomes.

The cornification is an essential process to build the skin barrier, during which the cell membrane of keratinocytes is displaced by the cornified envelope. The cornified envelope is an agglomeration of several structural proteins that are crosslinked. Cornifin-B and Fillagrin-2 are both parts of the cornified envelope and their upregulation indicates that ceramide NS may trigger the formation of the cornified envelope and therefore may improve the skin barrier function.

Desquamation is the last step of the keratinocytes terminal differentiation and Kallikrein-5 is a protease that cleaves the corneodesmosomes (cell-cell-adhesion of corneocytes) allowing the corneocytes to desquame.

Accordingly, in a further aspect the present invention relates to the use of ceramide NS for increasing keratinocyte terminal differentiation in the epidermis and/or improving skin barrier function in skin of an individual.

In some embodiments, topical compositions according to the present invention may be used for medical treatment of skin of an individual. In the context of the use of the inventive topical composition for medical or pharmaceutical purposes, the topical composition according to the invention may be referred to as a topical pharmaceutical composition or a pharmaceutical composition.

The pharmaceutical composition is for use in treating of a skin disease or of a skin pathological condition in an individual.

The term “skin disease” according to the invention is a disorder of structure or function in a human or animal that produces specific symptoms or that affects a specific location. i.e. skin, and is not simply a direct result of physical injury. Non-limiting examples of skin diseases include acne, rosacea, erythrocouperosis, psoriasis, xeroderma, ichthyosis, vascular disorders, diaper rash, atopic dermatitis, eczema, contact dermatitis, irritant dermatitis, allergic dermatitis, seborrheic dermatitis. The term “pathological skin condition” refers to abnormal anatomical or physiological conditions and objective or subjective manifestations of disease, not classified as disease or syndrome. Non-limiting example of pathological skin conditions include: rosacea, which is characterized by small, red, pus-filled bumps on the face and vitiligo, which results in large, irregular patches of skin.

A pharmaceutical composition of the present invention may in some embodiments consist of one or more compounds of formula I of the inventive topical composition (as any of the described above).

In other embodiments, the topical pharmaceutical composition of the present invention may further include an additional ingredient that is pharmaceutically active for treating a skin disease or a pathological skin condition.

Non-limiting examples of such additional pharmaceutical active agents include anti-acne agents, agents used to treat rosacea, analgesics, anesthetics, anorectals, antihistamines, anti-inflammatory agents including non-steroidal anti-inflammatory drugs, antibiotics, antifungals, antivirals, antimicrobials, anti-cancer actives, scabicides, pediculicides, antineoplastics, antiperspirants, antipruritics, antipsoriatic agents, antiseborrheic agents, biologically active proteins and peptides, burn treatment agents, cauterizing agents, depigmenting agents, depilatories, diaper rash treatment agents, enzymes, hair growth stimulants, hair growth retardants including DFMO and its salts and analogs, hemostatics, kerotolytics, canker sore treatment agents, cold sore treatment agents, dental and periodontal treatment agents, photosensitizing actives, skin protectant/barrier agents, steroids including hormones and corticosteroids, sunburn treatment agents, sunscreens, transdermal actives, nasal actives, vaginal actives, wart treatment agents, wound treatment agents, wound healing agents, etc.

Topical compositions of the invention may be advantageously administered onto skin of an individual for at least 7 days, such as for at least 14 days or for at least 21 days. For example, the topical composition is administered for at least 28 days. In practice, there is no time limitation for the administration period of a cosmetic compositions of the invention. For pharmaceutical composition of the invention, the administration period would be typically determined by a medical practitioner for each individual patient.

A topical composition (either cosmetic or pharmaceutical) may be administered one time or several times per day, such as 2-3 times per day.

Topical compositions of the present invention typically include or are incorporated into different types of vehicles and carriers. In some embodiments, the topical composition of the present invention further comprises a topically acceptable carrier. The vehicle or carrier can be a pharmaceutically and/or dermatologically acceptable vehicle or carrier. Non-limiting examples of vehicles or carriers include water, glycerin, alcohol, oil, a silicon containing compound, a silicone compound, and wax. Variations and other appropriate vehicles will be apparent to the skilled artisan and are appropriate for use in the present invention. In certain aspects, the concentrations and combinations of the compounds, ingredients, and agents can be selected in such a way that the combinations are chemically compatible and do not form complexes which precipitate from the finished product.

In some embodiments, a topical composition may contain a C₆-C₁₀₀ester base which functions as an acceptable carrier for the one or more compounds of formula I. Suitable examples of ester bases include:
(1) a C₇-C₆₀ neoalkanol ester. These esters may be formed from the reaction between C₁-C₂₂ alkanoic acid with neopentyl alcohol, neopentyl glycol, 2-butyl-2-ethyl-1,3-propanediol, 2,2,4-1rimethyl-1,3-pentane diol, trimethylol propane, pentaerythritol, di-trimethylol propane, di-pentaerythritol or pentaerythritol-trimethylol propane dimers. Examples include isostearyl neopentanoate, palmityl neopentanoate, tetraoctylpentaerythritol and diisopropyl neopentanoate. Amounts of this ester may suitably range from 1% to 90%, preferably from 20% to 75%, optimally from 30% to 50% by weight of the topical composition.
(2) a C₁₂-C₄₀ fatty glyceride ester alkoxylated with from 1 to 100 moles C₂-C₃ alkylene oxide per mole of glyceride. The C₁₂-C₄₀ fatty glyceride ester alkoxylate preferably includes 4 to 20 moles alkylene oxide per mole of glyceride. Preferred alkylene oxides are ethylene oxide and propylene oxide. Examples are PEG-6 caprylic/capric glyceride and PEG-8 caprylic/capric glyceride each of which are polyethylene glycol derivatives of a mixture of mono, di and triglycerides of caprylic and capric acids with a respective 6 and 8 moles of ethylene oxide. Amounts of this ester may suitably range from 1% to 90%, preferably from 5% to 50%, optimally from 10% to 20% by weight of the topical composition.
(3) a polyglycerol C₈-C₂₂ mono or di-fatty acid ester. Examples include polyglycerol-3 beeswax, polyglycerol-4 cocoate, polyglycerol-10 decalinoleate, polyglycerol-10 decaoleate, polyglycerol-7 decastearate, polyglycerol-2 diisostearate, polyglycerol-3 diisostearate, polyglycerol-7 diisostearate, polyglycerol-2 dioleate, polyglycerol-3 dioleate, polyglycerol-6 dioleate, polyglycerol-10 dioleate, polyglycerol-3 distearate, polyglycerol-6 distearate, polyglycerol-10 distearate, polyglycerol-10 heptaoleate, polyglycerol-10 heptastearate, polyglycerol-6 hexaoleate, polyglycerol-2 isostearate, polyglycerol-4 isostearate, polyglycerol-6 isostearate, polyglycerol-10 laurate, polyglycerol-10 myristate, polyglycerol-2 oleate, polyglycerol-3 oleate, polyglycerol-4 oleate, polyglycerol-6 oleate, polyglycerol-8 oleate, polyglycerol-10 oleate and combinations thereof. Most preferred is polyglycerol-6 dioleate which is a diester of oleic acid and a glycerin polymer containing an average of 6 glycerin units. Amounts of this ester may range from 1% to 50%, preferably from 5% to 25%, optimally from 10% to 20% by weight of the topical composition.
(4) a C₈-C₂₂ alkanol ester of a C₈-C₂₂ fatty acid. Non-limiting examples include cetyl octanoate, lauryl pentanoate, palmityl palmitoate, isostearyl decanoate, oleyl heptanoate and combinations thereof. Amounts of this material may range from 1% to 50%, preferably from 5% to 30%, optimally from 10% to 20% by weight of the topical composition.

The ester base may range from about 30% to 95%, preferably from 40% to 80%, optimally from 50% to 70% by weight of the topical composition.

The topical compositions may further contain a solubiliser in the form of a glycerol C₈-C₂₂ mono fatty acid ester. Non-limiting examples of solubilisers include glycerol monoisostearate, glycerol monobehenate and glycerol monopalmitate. The solubiliser may comprise about 0,1% to 20%, preferably from 0,5% to 10%, optimally from 1% to 5%, by mass of the topical composition.

The topical compositions may contain other carriers such as silicone oils. The silicone oils may be cyclic or linear polydimethylsiloxanes containing from 3 to 9, preferably from 4 to 5, silicon atoms, or polyalkyl siloxanes, polyalklyaryl siloxanes and polyether siloxane copolymers. Silicones may be present in amounts generally ranging from 1% to 50%, preferably from 2% to 25%, optimally between 10% and 20% by weight of the topical composition.

The topical compositions may also include additional ingredients typically found in cosmetic and skin care formulations such as cosmetic ingredients and pharmaceutical active ingredients.

Examples of further inclusions in topical compositions of the invention in different embodiments may include chalk, talc, fullers, earth, kaolin, starch, smectites clays, chemically modified magnesium aluminum silicate, organically modified montmorillonite clay, hydrated aluminum silicate, fumed silica, aluminum starch octenyl succinate. Also surfactants may be added including alkyl sulfates, such as Sodium Lauryl Sulfate, Sodium Laureth Sulfate, Sodium Coco-Sulfate and Sodium Trideceth Sulfate, betaines, such as cocobetaine, and carbohydrates, such as Lauryl Glucoside and Coco-Glucoside. Other ingredients are colouring agents, opacifiers and perfumes. Amounts of these adjunct materials may conveniently range anywhere from 0,001% up to 20% by weight of the composition.

The CTFA International Cosmetic Ingredient Dictionary and Handbook (2004 and 2008) describes a wide variety of non-limiting cosmetic ingredients that can be used in the context of the present invention. Examples of these ingredient classes include: fragrance agents (artificial and natural; e.g., gluconic acid, phenoxyethanol, and triethanolamine), dyes and color ingredients (e.g., Blue 1, Blue 1 Lake, Red 40, titanium dioxide, D&C blue no. 4, D&C green no. 5, D&C orange no. 4, D&C red no. 17, D&C red no. 33, D&C violet no. 2, D&C yellow no. 10, and D&C yellow no. 11), flavoring agents/aroma agents (e.g., Stevia rebaudiana (sweetleaf) extract, and menthol), adsorbents, lubricants, solvents, moisturizers (including, e.g., emollients, humectants, film formers, occlusive agents, and agents that affect the natural moisturization mechanisms of the skin), water-repellants, UV absorbers and/or reflectors (physical and chemical absorbers such as para-aminobenzoic acid (“PABA”) and corresponding PABA derivatives, titanium dioxide, zinc oxide, etc.), essential oils, vitamins (e.g., A, B, C, D, E, and K), trace metals (e.g., zinc, calcium and selenium), anti-irritants (e.g., steroids and non-steroidal anti-inflammatoires), botanical extracts (e.g., Aloe vera, chamomile, cucumber extract, Ginkgo biloba, ginseng, and rosemary), anti-microbial agents, antioxidants (e.g., BHT and tocopherol), chelating agents (e.g., disodium EDTA and tetrasodium EDTA), preservatives (e.g., methylparaben and propylparaben), pH adjusters (e.g., sodium hydroxide and citric acid), absorbents (e.g., aluminum starch octenylsuccinate, kaolin, corn starch, oat starch, cyclodextrin, talc, and zeolite), skin bleaching and lightening agents (e.g., hydroquinone and niacinamide lactate), humectants (e.g., sorbitol, urea, methyl gluceth-20, saccharide isomerate, and mannitol), exfoliants, waterproofing agents (e.g., magnesium/aluminum hydroxide stearate), skin conditioning agents (e.g., aloe extracts, allantoin, bisabolol, ceramides, dimethicone, hyaluronic acid, biosaccharide gum-1, ethylhexylglycerin, pentylene glycol, hydrogenated polydecene, octyldodecyl oleate, and dipotassium glycyrrhizate). Non-limiting examples of some of these ingredients are provided in the following subsections.

The topical compositions may also include fatty alcohols and fatty acids having from 10 to 20 carbon atoms. Especially preferred are such compounds as cetyl, myristyl, palmityl, isostearyl and stearyl alcohols and acids.

The topical compositions may further include α-hydroxy carboxylic acids. Suitable examples include: α-hydroxyethanoic acid, α-hydroxypropanoic acid, α-hydroxyhexanoic acid, α-hydroxyoctanoic acid, α-hydroxydecanoic acid, α-hydroxydodecanoic acid, α-hydroxytetradecanoic acid, α-hydroxyhexadecanoic acid, α-hydroxyoctadecanoic acid, α-hydroxyeicosanoic acid, α-hydroxydocosanoic acid, α-hydroxyhexacosanoic acid, and α-hydroxyoctacosanoic acid. The α-hydroxy carboxylic acid may be in the acid form or in salt form. Typical salts are the alkalimetal, ammonium and C₂-C₃₀ ammonium salts, for example the sodium, potassium, triethanolammonium and ammonium salts form. Typical salts are the alkalimetal, ammonium and C₂-C₃₀ ammonium salts, for example the sodium, potassium, triethanolammonium and ammonium salts. The α-hydroxy carboxylic acid will generally be present in a range from 0,001% to 20%, preferably from 0,01% to 15%, optimally from 0,5% to 10% by weight of the topical composition.

The topical composition may also include vitamins. Examples include sources or derivatives of vitamin C, such as ascorbyl palmitate, ascorbyl glucoside, magnesium ascorbyl phosphate, ascorbyl lysostearate, vitamin A palmitate (retinyl palmitate) and vitamin E linoleate (tocopheryl linoleate). Other esters of vitamin C, vitamin A and vitamin E may also be utilised.

The topical compositions may also include typical emulsifiers. The emulsifiers may be nonionic, anionic, cationic or amphoteric in nature. Examples of suitable emulsifiers include polyoxypropylene-polyoxyethylene ethers of cetyl alcohols and glycerol monoisostearate. The emulsifiers may suitably range from 0,5% to 30%, preferably from 1% to 15%, optimally from 3% to 8% by weight of the topical composition.

The topical compositions may also include thickeners. Examples include cross-linked polyacrylate materials and gums such as xanthan, carrageenan, gelatine, karaya, pectin and locust beans gum. The thickener may be present in amounts from about 0,1% to 20% by weight, preferably from 0,5% to 10% by weight, of the topical composition.

The topical compositions may also contain suitable preservatives. Suitable preservatives include alkyl esters of p-hydroxybenzoic acid, phenoxyethanol, hydantoin derivatives, proprionate salts, and a variety of quaternary ammonium compounds. For example, methyl paraben, propyl paraben, imidazolidinyl urea, sodium dehydroxyacetate and benzyl alcohol. Preservatives will usually be employed in amounts ranging from 0,1% to 2% by weight of the topical composition.

The topical composition may also include substances such as p-anisic acid, sodium anisate, levulinic acid, sodium levulinate, sodium hydroxide, caprylyl glycol, 1,2-hexanediol, pentylene glycol, glyceryl caprylate, glyceryl caprate, ethylhexylglycerin, Undecylenamidopropyl-trimonium Methosulfate, propylene glycol, phenylpropanol, phenethyl alcohol, methylpropanediol, Dipropylene Glycol, or a combination thereof.

In some embodiments, the present invention relates to a method for increasing the ceramide content of the stratum corneum, the method comprising topically administering a cosmetic composition of the present invention onto skin of an individual.

In some embodiments, the present invention relates to a method of improving skin barrier function, the method comprising topically administering a cosmetic composition of the present invention onto skin of an individual.

In some embodiments, the present invention relates to a method of increasing keratinocyte terminal differentiation in the epidermis, the method comprising topically administering a cosmetic composition of the present invention onto skin an individual.

In some embodiments, the present invention relates to a method of upregulating enzymes in the epidermis associated with epidermal ceramide synthesis, the method comprising topically administering a cosmetic composition of the present invention onto skin of a subject.

The methods may in some embodiments be for improving skin complexion, improving skin radiance, improving skin tone, skin soothing, reducing premature skin ageing, wrinkle smoothing and/or supporting healthy look of skin.

In one preferred embodiment, the present invention relates to a method for non-pharmacological management of a skin disease, such as aphotic dermatitis, or condition associated with an impaired skin barrier function, such as increased transepidermal water loss (TEWL), dry skin, rough skin, irritated skin, the method comprising topically administering a composition comprising one or more compounds of formula I, in particular at least one said compound that has lactosyl or glycosyl residue in the position X of formula I, onto a skin of an individual. In another preferred embodiment, the invention related to non-pharmacological intervention for mitigation of a skin condition or a skin disease in an individual who has, or who is susceptible to developing an impaired skin barrier function, the method comprising topically administering a composition comprising one or more compounds of formula I, in particular at least one said compound that has lactosyl or glycosyl residue in the position X of formula I, onto a skin of said individual. The individual who is susceptible to an impaired skin barrier function according to the invention is an individual has either
heredity of impaired skin barrier function, or
been exposed to hazardous environmental factors.

The relevant hazardous environmental factors include, but not limited to, climatic conditions, such as UV light or chemical pollutants, such as polycyclic aromatic pollutants (PAH), particulate matter PM2.5, blue light, ozone or cigarette smoke.

The invention in general relates to an individual who is a mammal, e.g. a domestic animal, like a dog or cat, or a human individual. Preferably, the individual is a human.

In some preferred embodiments, the human individual is an adult individual, i.e. a human of 20+ years old; in other preferred embodiments, the human individual may be a child or a teenager. The later may be preferred, e.g., when compositions of the invention are intended for prophylactic treatment, such as non-pharmacological intervention for mitigation of a skin condition or a skin disease in an individual who has, or who is susceptible to developing an impaired skin barrier function.

In the context of the invention it has been further found that application of any of ceramide NP, ceramide NG, glucosyl ceramide or lactosyl ceramide on skin leads to an upregulation of proteins of the mitochondrial respiratory chain.

In the context of the invention it has been further found that application of ceramide NP on the skin leads to an upregulation of NADH dehydrogenase [ubiquinone] iron-sulfur protein 8 (mitochondrial) and of phosphomevalonate kinase.

In the context of the invention it has been further found that application of ceramide NG on the skin leads to an upregulation of ATP synthase subunit g. (mitochondrial), of ATP synthase subunit delta (mitochondrial), of cytochrome c oxidase subunit 6C and of phosphomevalonate kinase.

In the context of the invention it has been further found that application of glucosyl ceramide NS on the skin leads to an upregulation of ATP synthase subunit g. (mitochondrial), of ATP synthase subunit delta (mitochondrial), of cytochrome c oxidase subunit 6C, of NADH dehydrogenase [ubiquinone] iron-sulfur protein 2 (mitochondrial), of NADH dehydrogenase [ubiquinone] 1 alpha subcomplex subunit 8, of ATP-dependent zinc metalloprotease YME1L1, of NAD-dependent malic enzyme (mitochondrial) and of phosphomevalonate kinase.

In the context of the invention it has been further found that application of lactosyl ceramide NS on the skin leads to an upregulation of ATP synthase subunit g. (mitochondrial), of ATP synthase subunit delta (mitochondrial), of cytochrome c oxidase subunit 6C, of cytochrome c oxidase subunit 2, of cytochrome c oxidase subunit NDUFA4, of cytochrome b-c1 complex subunit 9, of ATP-dependent zinc metalloprotease YMEIL1, of NAD-dependent malic enzyme (mitochondrial) and of phosphomevalonate kinase.

ATP is a versatile biochemical form of energy used to fuel metabolic processes. ATP is generated by the respiratory chain. During age and stress energy production is decreased and metabolism slowed down. Repair mechanisms are weakened and damage is accelerated. Activating the respiratory chain and therefore ATP generation keeps the metabolism high and provides energy for repair mechanisms, cells stay healthy & functional resulting in a healthy skin with a radiant complexion. Furthermore, an activated metabolism supports skin regeneration and helps to fight against signs of ageing.

Accordingly, ceramide NP, ceramide NG, glucosyl ceramide and/or lactosyl ceramide, or compositions comprising thereof may also be used for boosting energy of skin cells according to the invention.

Boosting energy of the skin cells as used herein refers to an improvement of overall metabolism and ATP-production and may also be referred to as skin vitalisation or skin energizing. Different aspects of the invention discussed above are described in the following selected, but not limiting, embodiments:
1. Topical composition comprising one or more compounds of formula I

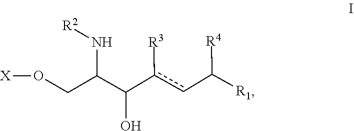

wherein
X is hydrogen or a glycosyl moiety,
the carbon-carbon bond noted [symbol] is a double or a single bond,
R¹ is an alkyl chain having 10-20 carbon atoms,
R² is hydrogen or a substituted or unsubstituted acyl having 16-34 carbon atoms,
R³ is hydrogen when the carbon-carbon bond noted [symbol] is a double or a single bond, and R³ is —OH when the carbon-carbon bond noted [symbol] is a single bond, R⁴ is hydrogen when the carbon-carbon bond noted [symbol] is a double or a single bond, and R⁴ is hydrogen or —OH when the carbon-carbon bond noted [symbol] is a double bond,
said composition is characterized in that at least one of the one or more compounds of formula I has a lactosyl moiety or a glucosyl moiety in the position X.
2. Topical composition according to embodiment 1, further comprising at least one compound of formula I wherein X is galactosyl and/or hydrogen.
3. Topical composition according to embodiment 1 or 2, wherein for one or more of the compounds of formula I, the stereochemical configuration is (2S,3R,4E) and the carbon-carbon bond noted [symbol] is a double bond.
4. Topical composition according to embodiment 1 or 2, wherein for one or more of the compounds of I, the stereochemical configuration is (2S,3S,4R) or (2S,3S), and the carbon-carbon bond noted [symbol] is a single bond.
5. Topical composition according to any one of embodiments 1 to 4, wherein for one or more of the compounds of formula I, R¹ is —C₁₂H₂₅ and/or R² is selected from hexadecanoyl, 2-hydroxyhexadecanoyl, octadecanoyl, 2-hydroxyoctadecanoyl, eicosanoyl, 2-hydroxyeicosanoyl, lignoceroyl, 2-hydroxytetracosanoyl, 30-(linoleyloxy)-triacontanoyl.
6. Topical composition according to any one of embodiments 1 to 5, wherein the one or more compounds of formula I are present in an amount of about 0,0001% to about 10% wt/wt, such as from about 0,005% to about 5% wt/wt, or from about 0,01% to about 1% wt/wt.
7. Topical composition according to any one of embodiments 1 to 6, having a pH in the range of from about 4.5 to about 8.
8. Topical composition according to any one of embodiments 1 to 7, wherein the topical composition is formulated as an emulsion, an oil, a toner, a conditioner, a scalp lotion or a shampoo, and/or for application on the face, body and/or hair.
9. Topical composition according to any one of embodiments 1 to 8, wherein the composition comprises a compound of I which is ceramide NS, ceramide NG, ceramide NP, glucosyl ceramide or lactosyl ceramide.
10. Topical composition according to any one of embodiments 1 to 9, wherein the compound of I is a compound of Ia to Ie:

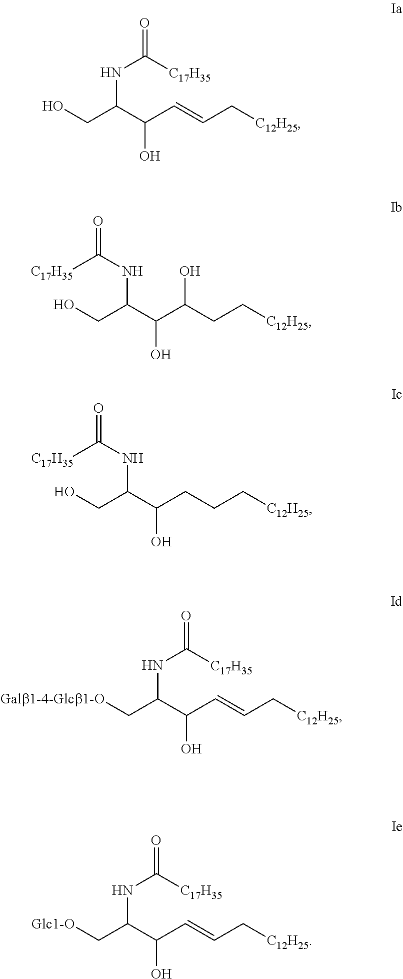

11. Topical composition according to any one of embodiments 1 to 10, wherein the compound of I is a compound of Iaa to Iee:

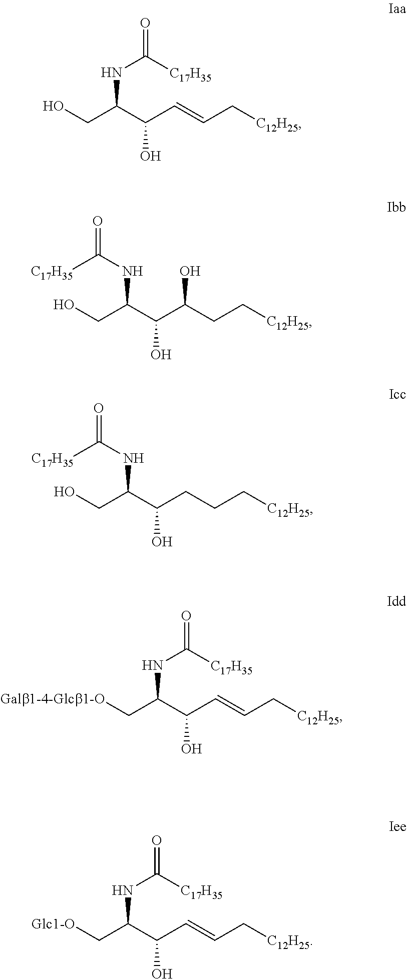

12. Topical composition according to any one of embodiments 1 to 11, wherein the composition
   increases the ceramide content of the stratum corneum,
   increases keratinocyte terminal differentiation in the epidermis,
   upregulates enzymes in the epidermis associated with epidermal ceramide synthesis, and/or
   improves skin barrier function
   in skin of an individual.
13. A topical cosmetic composition comprising a composition according to any one of embodiments 1-12, for use in cosmetic treatment of skin of an individual, wherein said treatment improves the visual appearance of skin of said individual.
14. A cosmetic topical composition of embodiment 13, wherein the skin of the individual is aged and/or dry.
15. Use of lactosyl ceramide for
   increasing the ceramide content of the stratum corneum,
   increasing keratinocyte terminal differentiation in the epidermis,
   upregulating enzymes in the epidermis associated with epidermal ceramide synthesis, and/or
   improving skin barrier function
in skin of an individual.
16. Use of ceramide NP for
   increasing the ceramide content of the stratum corneum,
   increasing keratinocyte terminal differentiation in the epidermis,
   upregulating enzymes in the epidermis associated with epidermal ceramide synthesis, and/or
   improving skin barrier function
in skin of an individual.
17. Use of ceramide NG for
   increasing the ceramide content of the stratum corneum,
   increasing keratinocyte terminal differentiation in the epidermis,
   upregulating enzymes in the epidermis associated with epidermal ceramide synthesis, and/or
   improving skin barrier function
in skin of an individual.
18. Use of glucosyl ceramide for
   increasing the ceramide content of the stratum corneum,
   upregulating enzymes in the epidermis associated with epidermal ceramide synthesis, and/or
   improving skin barrier function
in skin of an individual.
19. Use of ceramide NS for
   increasing keratinocyte terminal differentiation in the epidermis and/or
   improving skin barrier function
in skin of an individual.
20. Use according to any one of embodiments 15 to 19, wherein the cosmetic treatment has one or more of following beneficial effects on skin of the individual selected from
   Improving skin complexion
   Improving skin radiance
   Improving skin tone
   Skin soothing
   Reducing premature skin ageing
   Wrinkle smoothing
and/or
   Supporting healthy look of skin.
21. A topical pharmaceutical composition according to any one of embodiments 1 to 13 for use in treating a skin pathological condition or a skin disease in an individual.
22. A topical pharmaceutical composition comprising a composition according to embodiment 21, further comprising one or more compounds that are
   structurally different from a compound of formula I, and/or
   pharmaceutically active for treating of a skin disease or a pathological skin condition.
23. A topical pharmaceutical composition according to embodiment 21 or 22, for use in the treatment of a skin disease selected from acne, ichthyosis, xeroderma, eczema, atopic dermatitis and/or psoriasis.
24. Method for non-pharmacological management of a skin condition associated with an impaired skin barrier function, such as increased transepidermal water loss (TEWL), dry skin, rough skin, irritated skin, the method comprising topically administering a composition of any one of embodiments 1 to 13 onto a skin of an individual.
25. Method for non-pharmacological intervention for mitigation of a skin condition or a skin disease in an individual who has, or who is susceptible to developing an impaired skin barrier function, the method comprising topically administering a composition of any one of embodiments 1 to 13 onto a skin of said individual.
26. Method according to embodiment 25, wherein the individual who is susceptible to an impaired skin barrier function has
   heredity of impaired skin barrier function, or
   been exposed to hazardous environmental factors.
27. Method according to embodiment 26, wherein the hazardous environmental factors include climatic conditions, such as UV light or chemical pollutants, such as polycyclic aromatic pollutants (PAH), particulate matter PM2.5, blue light, ozone or cigarette smoke.
28. Method according to embodiment 25, wherein the skin disease is atopic dermatitis.
29. Method according to any of embodiments 13-28, wherein the individual is a mammal, preferably a human.
30. Method according to embodiment 29, wherein the human individual is a child or a teenager.
31. Method according to any one of embodiments 21 to 30, wherein the topical composition is administered onto skin of an individual for at least 7 days, such as for at least 14 days.

### Examples

The examples described below are for illustration purposes only. The invention is not limited in scope to embodiments described in the examples.

### Example 1: Topical Compositions Comprising Ceramide Blends

| | Total | | | | | |
|---|---|---|---|---|---|---|
| | ceramide | | | | | |
| | content | | Ceramide | Ceramide | Ceramide | |
| | (wt/wt) | LacCNS | NP | NS | NG | GlcCNS |
| 1. | 0.1% | 0.08 | | | | 0.02 |
| 2. | 0.2% | 0.04 | 0.16 | | | |
| 3. | 0.2% | 0.04 | | 0.16 | | |
| 4. | 0.2% | 0.04 | | | 0.16 | |

### Example 2: Topical Compositions Comprising Ceramide Blends

| | Total ceramide | Ceramide | Ceramide | Ceramide | |
|---|---|---|---|---|---|
| | content (wt/wt) | NP | NS | NG | GlcCNS |
| 5. | 0.1% | 0.06 | 0.02 | 0.03 | |
| 6. | 0.2% | 0.11 | 0.04 | 0.05 | |
| 7. | 0.5% | 0.28 | 0.09 | 0.13 | |
| 8. | 0.1% | 0.075 | 0.02 | 0.005 | |
| 9. | 0.2% | 0.15 | 0.04 | 0.01 | |
| 10. | 0.2% | 0.1 | 0.05 | | 0.05 |

### Example 3

Human epidermal keratinocytes are obtained from a commercial vendor. The cells are isolated from juvenile foreskin or from adult normal human tissue collected from various sites that include face, breast, abdomen, and thigh. The cells are cultivated in a T25 flask using a commercial keratinocyte Growth Medium which is a serum-free medium and optimized for the in vitro cultivation of epidermal keratinocytes without feeder cells. A commercial growth supplement and calcium is added to a final concentration of 1.2 mM to induce differentiation.

Upon reaching cell confluence, the cells are treated with either 200 ppm glucosylceramide or 200 ppm lactosylceramide, each dissolved in dimethyl sulfoxide (DMSO) for 1 week. The cells are homogenised with 2 mL chloroform:methanol (2:1), and transferred to a vial containing Phosphate Buffered Saline (PBS). The homogeniser is rinsed twice with solvent and all rinses are together. The organic phase is evaporated to dryness.

Ceramide levels produced by the keratinocytes are measured by a modified high-performance thin layer chromatography (HPTLC) method. The dried residue is dissolved in 200 μL chloroform:methanol (2:1). Samples of 80 μl and 20 μL are then subjected to a HPTLC protocol in which the HPTLC plates are developed using a sequential development system: (1) dichloromethane:ethylacetate:acetone (80:16:4) to 85 mm, (2) chloroform:methanol:acetone (76:16:8) to 80 mm, (3) hexane:chloroform:acetic acid:acetone:methanol (6:80:0,1:10:4) to 85 mm. The plates are stained with 3% copper acetate in 8% phosphoric acid and charred at 160° C. for the quantitation of ceramides. Quantification is performed against known quantities of Ceramide NP by laser scanning densitometry. Each plate is scanned at 425 nm and quantified using WinCATS software.

Gene expression is determined by isolating RNA samples from the keratinocytes using a Qiagen RNeasy kit with DNase I digestion. Reverse transcription is performed using a high-capacity cDNA kit, cDNA samples of 40 ng are used for qPCR. Taqman gene expression assay is purchased commercially, and the qPCR reaction is performed using a fast amplifier. Data are normalized by reference genes, either polymerase (RNA) II polypeptide A (POLR2A) and ribosomal protein large, PO (RPLPO) of which expression does not change by the treatment. Relative gene expression is calculated by comparative CT method.

Up-regulation of key enzymes catalysing ceramide synthesis such as β-glucocerebrosidase (GBA) is found in keratinocytes treated with glucosylceramide or lactosylceramide. Further the expression of the ABCA12 gene increases. This gene encodes a protein responsible for ceramide trafficking in keratinocytes.

Total ceramides in keratinocytes treated glucosylceramide or lactosylceramide are elevated compared to untreated keratinocytes. The differentiation marker genes, involucrin and transglutaminase-1, increase in expression. This response is also observed in additional genes of keratinocyte differentiation as SPRR1 and SPRR2. In addition, the keratinocytes treated with glucosylceramide or lactosylceramide display reduced gene expression of inflammatory cytokines such as IL8 and TNF.

### Example 4: Characterisation of Effects of (Glycosylated) Ceramides and (Glyco)Sphingolipids as Cosmetic Active Ingredients

Material and methods: Reconstructed Human Epidermis (RHE) cell culture was used, as this system mimics the in vivo 3D structure of epidermal tissue as well as the conditions and processes that occur in normal epidermis. RHE were cultured using the medium provided by the manufacturer until analysis. RHE were maintained in survival for 24 hours, before RHE were topically treated with lipid in a base emulsion for 24h. 3 RHE were treated with base emulsion alone and 3 more remained untreated as a control batch (Ctrl). RHE were incubated under classical cell culture conditions (37° C., 5% CO2).

Sample preparation and LC-MS/MS analysis: Protein was extracted using the Pierce Total Protein Extraction Kit (Thermo Fisher) according to manufacturer's recommendations. The protein was mixed with Laemmli buffer and heated to 37° ° C. for 30 min. Protein concentration was determined by the BCA method and standardized for all samples. The samples were separated by SDS-PAGE and digested with trypsin overnight. The peptides generated were acidified and separated. The mass spectra were queried using Proteome Discoverer (version 2.5). The resulting MS/MS data were queried against the Homo sapiens proteome UP000005640 (20371 reviewed entries). Results were filtered based on unique peptides >2, and global peptide scores. Analyses were performed using Proteome Discoverer 2.5 to check for overall sample quality.

Bioinformatics and statistical analysis: Proteomic analysis returned a mean total of 34961 high-quality peptides corresponding to a mean 2422 identifiable and quantifiable RHE proteins among conditions. A summary of proteins differentially expressed within conditions is provided in Annexe 3. Proteins with a p-value≤0,05 in at least one of the concentrations tested for a lipid were considered significant and used for pairwise comparisons. Relevant proteins were classified by their biological processes and associated pathways using the publicly available gene ontology (GO) database provided by the Gene Ontology Consortium and Reactome. Interactome analysis was performed using Cytoscape combined with STRING.

1. Topical composition comprising one or more compounds of formula I 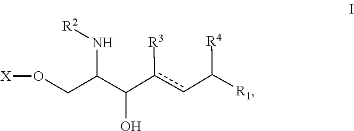
wherein
X is hydrogen or a glycosyl moiety,
the carbon-carbon bond noted [symbol] is a double or a single bond,
R¹ is an alkyl chain having 10-20 carbon atoms,
R² is hydrogen or a substituted or unsubstituted acyl having 16-34 carbon atoms,
R³ is hydrogen when the carbon-carbon bond noted [symbol] is a double or a single bond, and R³ is —OH when the carbon-carbon bond noted [symbol] is a single bond,
R⁴ is hydrogen when the carbon-carbon bond noted [symbol] is a double or a single bond, and R⁴ is hydrogen or —OH when the carbon-carbon bond noted [symbol] is a double bond, said composition is characterized in that at least one of the one or more compounds of formula I has a lactosyl moiety or a glucosyl moiety in the position X.

2. Topical composition according to claim 1, further comprising at least one compound of formula I wherein X is galactosyl and/or hydrogen.

3. Topical composition according to claim 1 or 2, wherein for one or more of the compounds of formula I, the stereochemical configuration is (2S,3R,4E) and the carbon-carbon bond noted [symbol] is a double bond.

4. Topical composition according to claim 1 or 2, wherein for one or more of the compounds of I, the stereochemical configuration is (2S,3S,4R) or (2S,3S), and the carbon-carbon bond noted [symbol] is a single bond.

5. Topical composition according to any one of claims 1 to 4, wherein for one or more of the compounds of formula I, R¹ is —C₁₂H₂₅ and/or R² is selected from hexadecanoyl, 2-hydroxyhexadecanoyl, octadecanoyl, 2-hydroxyoctadecanoyl, eicosanoyl, 2-hydroxyeicosanoyl, lignoceroyl, 2-hydroxytetracosanoyl, 30-(linoleyloxy)-triacontanoyl.

6. Topical composition according to any one of claims 1 to 5, wherein the one or more compounds of formula I are present in an amount of about 0,0001% to about 10% wt/wt, such as from about 0,005% to about 5% wt/wt, or from about 0,01% to about 1% wt/wt.

7. Topical composition according to any one of claims 1 to 6, having a pH in the range of from about 4.5 to about 8.

8. Topical composition according to any one of claims 1 to 7, wherein the topical composition is formulated as an emulsion, an oil, a toner, a conditioner, a scalp lotion or a shampoo, and/or for application on the face, body and/or hair.

9. Topical composition according to any one of claims 1 to 8, wherein the composition comprises a compound of I which is ceramide NS, ceramide NG, ceramide NP, glucosyl ceramide or lactosyl ceramide.

10. Topical composition according to any one of claims 1 to 9, wherein the compound of I is a compound of Ia to Ie:

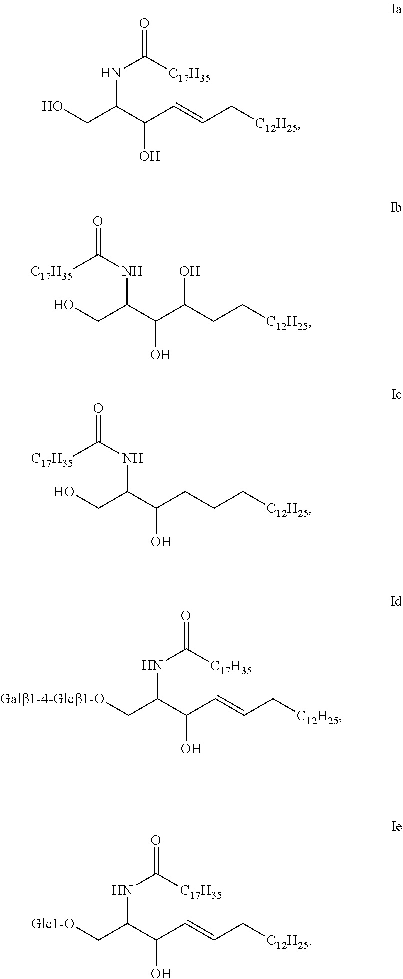

11. Topical composition according to any one of claims 1 to 10, wherein the compound of I is a compound of Iaa to Iee:

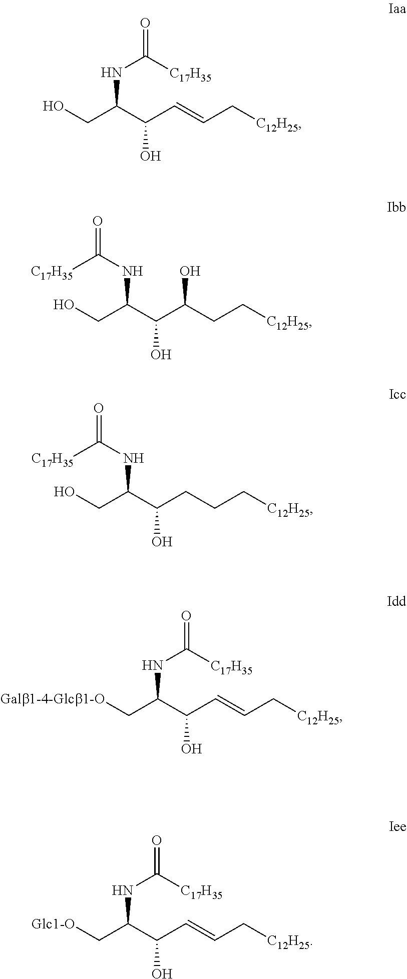

12. Topical composition according to any one of claims 1 to 11, wherein the composition
increases the ceramide content of the stratum corneum,
increases keratinocyte terminal differentiation in the epidermis,
upregulates enzymes in the epidermis associated with epidermal ceramide synthesis, and/or
improves skin barrier function
in skin of an individual.

13. A topical cosmetic composition comprising a composition according to any one of claims 1-12, for use in cosmetic treatment of skin of an individual, wherein said treatment improves the visual appearance of skin of said individual.

14. A cosmetic topical composition of claim 13, wherein the skin of the individual is aged and/or dry.

15. Use of lactosyl ceramide for
increasing the ceramide content of the stratum corneum,
increasing keratinocyte terminal differentiation in the epidermis,
upregulating enzymes in the epidermis associated with epidermal ceramide synthesis, and/or
improving skin barrier function
in skin of an individual.

16. Use of ceramide NP for
increasing the ceramide content of the stratum corneum,
increasing keratinocyte terminal differentiation in the epidermis,
upregulating enzymes in the epidermis associated with epidermal ceramide synthesis, and/or
improving skin barrier function
in skin of an individual.

17. Use of ceramide NG for
increasing the ceramide content of the stratum corneum,
increasing keratinocyte terminal differentiation in the epidermis,
upregulating enzymes in the epidermis associated with epidermal ceramide synthesis, and/or
improving skin barrier function
in skin of an individual.

18. Use of glucosyl ceramide for
increasing the ceramide content of the stratum corneum,
upregulating enzymes in the epidermis associated with epidermal ceramide synthesis, and/or
improving skin barrier function
in skin of an individual.

19. Use of ceramide NS for
increasing keratinocyte terminal differentiation in the epidermis and/or
improving skin barrier function
in skin of an individual.

20. Use according to any one of claims 15 to 19, wherein the cosmetic treatment has one or more of following beneficial effects on skin of the individual selected from
Improving skin complexion
Improving skin radiance
Improving skin tone
Skin soothing
Reducing premature skin ageing
Wrinkle smoothing
and/or
Supporting healthy look of skin.

21. A topical pharmaceutical composition according to any one of claims 1 to 13 for use in treating a skin pathological condition or a skin disease in an individual.

22. A topical pharmaceutical composition comprising a composition according to claim 21, further comprising one or more compounds that are
structurally different from a compound of formula I, and/or
pharmaceutically active for treating of a skin disease or a pathological skin condition.

23. A topical pharmaceutical composition according to claim 21 or 22, for use in the treatment of a skin disease selected from acne, ichthyosis, xeroderma, eczema, atopic dermatitis and/or psoriasis.

24. Method for non-pharmacological management of a skin condition associated with an impaired skin barrier function, such as increased transepidermal water loss (TEWL), dry skin, rough skin, irritated skin, the method comprising topically administering a composition of any one of claims 1 to 13 onto a skin of an individual.

25. Method for non-pharmacological intervention for mitigation of a skin condition or a skin disease in an individual who has, or who is susceptible to developing an impaired skin barrier function, the method comprising topically administering a composition of any one of claims 1 to 13 onto a skin of said individual.

26. Method according to claim 25, wherein the individual who is susceptible to an impaired skin barrier function has
heredity of impaired skin barrier function, or
been exposed to hazardous environmental factors.

27. Method according to claim 26, wherein the hazardous environmental factors include climatic conditions, such as UV light or chemical pollutants, such as polycyclic aromatic pollutants (PAH), particulate matter PM2.5, blue light, ozone or cigarette smoke.

28. Method according to claim 25, wherein the skin disease is atopic dermatitis.

29. Method according to any of claims 13-28, wherein the individual is a mammal, preferably a human.

30. Method according to claim 29, wherein the human individual is a child or a teenager.

31. Method according to any one of claims 21 to 30, wherein the topical composition is administered onto skin of an individual for at least 7 days, such as for at least 14 days.
